(19) United States
(12) Patent Application Publication
(10) Pub. No.: US 2016/0206211 A1
(43) Pub. Date: Jul. 21, 2016

(54) SURFACE SIMULATION

(71) Applicant: REAL IMAGING LTD., Lod (IL)

(72) Inventors: Eyal NAIMI, Bet-Shemesh (IL); Israel Boaz ARNON, Halamish (IL); Yoel ARIELI, Jerusalem (IL)

(21) Appl. No.: 14/915,296

(22) PCT Filed: Aug. 25, 2014
(86) PCT No.: PCT/IL2014/050759

§ 371 Date: Feb. 29, 2016

(30) Foreign Application Priority Data
Aug. 29, 2013 (GB) 1315375.4

(51) Int. Cl. A61B 5/01; A61B 5/00

(52) U.S. Cl. CPC A61B 5/015; A61B 5/0075; A61B 5/0077; A61B 5/4884; A61B 5/7264; A61B 5/0091; A61B 2576/02

(57) ABSTRACT

An imaging method comprising: receiving a spatial thermal representation of a curved body section, wherein the spatial thermal representation comprises a thermal image associated with spatial data; and generating a theoretical thermal simulation of the curved body section, wherein said generating of the theoretical thermal simulation is based on the spatial data of the representation and on predetermined thermodynamic logic of a type of the curved body section.

### FIELD OF THE INVENTION

The invention relates to surface simulation.

### BACKGROUND

The present invention, in some embodiments thereof, relates to IR (Infra Red) images and radiometric data, and, more particularly, but not exclusively, to creation by calculation i.e. by modeling and analysis of IR images, IR data and radiometric data.

The use of imaging in diagnostic medicine dates back to the early 1900s. Presently there are numerous different imaging modalities at the disposal of a physician allowing imaging of hard and soft tissues and characterization of both normal and pathological tissues.

Infrared cameras produce two-dimensional images known as IR (Infra Red) images. IR image is typically obtained by receiving from the body of the subject radiation at any one of several infrared wavelength ranges and analyzing the radiation to provide a two-dimensional radiometric map of the surface (i.e. temperature). The IR image can be in the form of either or both of a visual image and corresponding radiometric data.

U.S. Pat. No. 7,072,504 the contents of which are hereby incorporated by reference, discloses an approach which utilizes two infrared cameras (left and right) in combination with two visible light cameras (left and right). The infrared cameras are used to provide a three-dimensional thermographic image and the visible light cameras are used to provide a three-dimensional visible light image. The three-dimensional thermographic and three-dimensional visible light images are displayed to the user in an overlapping manner.

U.S. Pat. No. 7,292,719, the contents of which are hereby incorporated by reference discloses a system for determining presence or absence of one or more thermally distinguishable objects in a living body.

Also of interest is U.S. Pat. No. 6,442,419 discloses a scanning system including an infrared detecting mechanism which performs a 360° data extraction from an object, and a signal decoding mechanism, which receives electrical signal from the infrared detecting mechanism and integrates the signal into data of a three-dimensional profile of the object.

U.S. Pat. No. 6,850,862 discloses an apparatus which uses radiometric sensors to detect radiation from various layers within the object over a range of wavelengths from radio waves through the infrared.

U.S. Pat. No. 5,961,466 discloses detection of breast cancer from a rapid time series of infrared images which is analyzed to detect changes in the distribution of thermoregulatory frequencies over different areas of the skin.

### SUMMARY

The following embodiments and aspects thereof are described and illustrated in conjunction with systems, tools and methods which are meant to be exemplary and illustrative, not limiting in scope.

There is provided, in accordance with an embodiment, an imaging method comprising: receiving a spatial infra-red (IR) representation of a curved body section, wherein the spatial IR representation comprises a IR image associated with spatial data; and generating a calculated thermal simulation of the curved body section, wherein said generating of the theoretical thermal simulation is based on the spatial data of the representation and on predetermined thermodynamic logic of a type of the curved body section.

There is further provided, in accordance with an embodiment, an imaging system comprising: an imaging device; and a hardware data processor configured to: (a) generate a spatial thermal representation of a curved body section, wherein the spatial thermal representation comprises a thermal image associated with spatial data, and (b) generate a theoretical thermal simulation of the curved body section, wherein said generate of the theoretical thermal simulation is based on the spatial data of the representation and on predetermined thermodynamic logic of a type of the curved body section.

There is yet further provided, in accordance with an embodiment, an imaging method comprising: receiving spatial data of a curved body section; and generating a theoretical thermal simulation of the curved body section, wherein said generating of the theoretical thermal simulation is based on the spatial data of the representation and on predetermined thermodynamic logic of a type of the curved body section.

In some embodiments, the method further comprises receiving a spatial thermal representation of the curved body section, wherein the spatial thermal representation comprises said spatial data and a thermal image associated with said spatial data.

In some embodiments, the method further comprises comparing the spatial thermal representation and the theoretical thermal simulation.

In some embodiments, the method further comprises detecting an abnormality in the curved body section, wherein said detecting is based on said comparing of the spatial thermal representation and the theoretical thermal simulation.

In some embodiments, the method further comprises back-solving a parameter of the abnormality inside the curved body section.

In some embodiments, said back-solving comprises: generating a plurality of additional theoretical thermal simulations of a theoretical tumor inside the curved body section, wherein, in each simulation of the plurality of additional theoretical thermal simulations, a parameter of the theoretical tumor is adjusted; and comparing the spatial thermal representation and the plurality of additional theoretical thermal simulations, to determine which simulation of the plurality of additional theoretical thermal simulations is closest to the representation.

In some embodiments, the parameter of the abnormality is selected from the group consisting of: a location of the abnormality inside the curved body section, a size of the abnormality and a shape of the abnormality.

In some embodiments, said spatial thermal representation is responsive to a cold stress test, thereby enhancing a contrast between the abnormality and a normal tissue adjacent to the abnormality.

In some embodiments, the predetermined thermodynamic logic is under an influence of a theoretical cold stress test.

In some embodiments, the predetermined thermodynamic logic of the type of the curved body section is computed based on healthy subjects.

In some embodiments, the curved body section comprises one or more breasts.

In some embodiments, said hardware data processor is further configured to compare the spatial thermal representation and the theoretical spatial thermal simulation.

In some embodiments, said hardware data processor is further configured to detect an abnormality in the curved body section, wherein said detect is based on said comparing of the spatial thermal representation and the theoretical thermal simulation.

In some embodiments, said hardware data processor is further configured to back-solve a parameter of the abnormality inside the curved body section.

In some embodiments, said back-solve comprises: generating a plurality of additional theoretical thermal simulations of a theoretical tumor inside the curved body section; wherein, in each simulation of the plurality of additional theoretical thermal simulations, a parameter of the theoretical tumor is adjusted; and comparing the spatial thermal representation and the plurality of additional theoretical thermal simulations, to determine which simulation of the plurality of additional theoretical thermal simulations is closest to the representation.

In some embodiments, said imaging device comprises a thermal imaging device and a visible light imaging device.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A shows a three-dimensional spatial representation illustrated as a non-planar surface, in accordance with an embodiment;

FIG. 1B shows a thermographic image illustrated as planar isothermal contours, in accordance with an embodiment;

FIG. 1C shows a synthesized IR-spatial image formed by mapping the thermographic image on a surface of the three-dimensional spatial representation, in accordance with an embodiment;

FIG. 2 shows a flow chart of a method suitable for analyzing a thermal image of a body section, in accordance with an embodiment;

FIG. 3 shows a flowchart of another method suitable for analyzing a thermal image of a body section, in accordance with an embodiment;

FIG. 4 shows a flowchart of another method suitable for analyzing a thermal image of a body section, in accordance with an embodiment;

FIG. 5 shows a flowchart of another method suitable for analyzing a thermal image of a body section; in accordance with an embodiment;

FIG. 6A shows a schematic illustration of an IR-spatial imaging system, in accordance with an embodiment;

FIG. 6B shows an illustration of an operation principle of IR-spatial imaging system, in accordance with an embodiment;

FIG. 6C shows an illustration of another operation principle of IR-spatial imaging system, in accordance with an embodiment;

FIG. 7A shows an illustration of another operation principle of IR-spatial imaging system, in accordance with an embodiment;

FIG. 7B shows an illustration of another operation principle of IR-spatial imaging system, in accordance with an embodiment;

FIG. 7C shows an illustration of another operation principle of IR-spatial imaging system, in accordance with an embodiment;

FIG. 7D shows an illustration of another operation principle of IR-spatial imaging system, in accordance with an embodiment;

FIG. 7E shows an illustration of another operation principle of IR-spatial imaging system, in accordance with an embodiment;

FIG. 8A shows a pictorial view of a spatial thermal representation of breasts of a healthy subject;

FIG. 8B shows a pictorial view of a theoretical thermal simulation of the breasts of the healthy subject;

FIG. 8C shows a pictorial view of a comparison between the spatial thermal representation of the breasts of the healthy subject and the theoretical thermal simulation of the breasts of the healthy subject;

FIG. 9A shows a pictorial view of a spatial thermal representation of breasts of an unhealthy subject;

FIG. 9B shows a pictorial view of a theoretical thermal simulation of the breasts of the unhealthy subject; and

FIG. 9C shows a pictorial view of a comparison between the spatial thermal representation of the breasts of the unhealthy subject and the theoretical thermal simulation of the breasts of the unhealthy subject.

### DETAILED DESCRIPTION

An imaging method for generating a thermal simulation of a curved body section is disclosed herein. The present invention, in some embodiments thereof, relates to thermal images and, more particularly, but not exclusively, to the creation and analysis of IR images and thermal data.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In accordance with some embodiments, an imaging method may include generating, or receiving an already-generated spatial thermal representation of a curved body section, such as one or more female breasts. This spatial thermal representation includes a thermal (e.g. IR) image associated with spatial data of the curved body section. Then, a theoretical thermal simulation of the curved body section is generated, based on the spatial data of the representation and on predetermined thermodynamic logic of a type of the curved body section. The thermodynamic logic of a type of the curved body section may be, for example, the general thermodynamic behavior of a female's breasts. Advantageously, the thermodynamic logic is based on mathematical modeling of a general case of female breasts, constructed based on the thermodynamic behavior of breasts of healthy subjects.

In some embodiments, the spatial thermal representation and the theoretical thermal simulation are compared. Each of the spatial thermal representation and the theoretical thermal simulation may be constructed as a three-dimensional heat map, showing the temperature at different regions of the curved body section. Accordingly, their comparison may include deducting the theoretical thermal simulation from the spatial thermal representation, thereby obtaining a three-dimensional heat map of the thermal difference between the thermal behavior exhibited in reality by the curved body section and the theoretical thermal behavior of a healthy curved body section. The difference may be indicative of an abnormality in the curved body section, such as, for example, the existence of one or more tumors in the breast. The term “tumor”, as referred to herein, may relate to an abnormal mass of tissue, whether malignant, pre-malignant or benign.

In some embodiments, the method further includes back-solving a parameter of the abnormality inside the curved body section. The term “back solving”, as referred to herein, may relate to the computing method also known as “goal seeking”, which is often defined as the ability to calculate backward to obtain an input that would result in a given output. In the context of present embodiments, the output is the determination that a tumor exists in one or more of the breasts, as well as the particular representation of that tumor in the spatial thermal representation which was obtained. The purpose of the back solving may be to determine or at least estimate the real (or near-real) three-dimensional location, size, shape and/or density of the tumor inside the curved body section, based on its manifestation in the spatial thermal representation. Namely, the input sought by the back solving process is the actual location of the tumor inside the breast, whereas the output available is the manifestation of the tumor in the spatial thermal representation. The back solving may be further aimed at assessing the type of the abnormality, namely to categorize it as a benign or malignant tumor, and optionally, if the tumor is malignant, to determine its stage.

The back solving may be conducted as follows: first, the present method generates a plurality of additional theoretical thermal simulations of a theoretical tumor inside the curved body section. In other words, the method generates many (for example dozens, hundreds, thousands or more) possible inputs, each being of a theoretical abnormality (tumor) structured and positioned differently inside the curved body section. That is, a parameter of the abnormality is adjusted for each subsequent generation of an input. The parameters may be, for example, the location of the abnormality inside the curved body section, its shape and/or size.

Then, the method may compare the spatial thermal representation and the plurality of inputs (namely, the additional theoretical thermal simulations), to determine which input is the closest one to the representation. For example, it may be determined that a tumor characterized by a shape and a size A and located at coordinates B is the likely cause of the abnormality visualized in the spatial thermal representation.

In some embodiments, the subject may be subjected to a cold stress test prior to and/or during the acquisition of the thermal image and the spatial data. The cold stress test may include, for example, instructing the subject to hold a cold object, such as a container filled with a frozen liquid, in one or both hands. Accordingly, the resulting spatial thermal representation is responsive to the subject's body reaction to the cold stress test. The cold stress test may enhance the contrast between the abnormality and a normal tissue adjacent to the abnormality, since the cold may not influence the blood flow to the abnormality at a higher level than the decrease of blood flow to normal tissue adjacent to the abnormality.

In some embodiments, the method or at least parts thereof may be carried out by an imaging system which includes an imaging device and a hardware data processor. The processor may be configured to, for example (a) generate the spatial thermal representation and (b) generate the theoretical thermal simulation of the curved body section.

Embodiments of the present invention provide an approach which may enable the analysis of a thermal image, e.g., for the purpose of determining the likelihood that the image indicates presence of a thermally distinguishable region. When the thermal image is of a body section such as a breast of a woman, the analysis of the present embodiments may be advantageously used to extract properties of the underlying tissue. For example, determination of the likelihood that a thermally distinguished region is present in the body section may be used to for assessing whether or not the body section may have pathology such as a tumor.

The analysis according to some embodiments of the present invention may be based on surface information obtained from the surface of the body section. Generally, the measured surface information may be compared to a predicted or may calculate surface information. In some embodiments of the present invention the surface comparison may relate to the likelihood that a thermally distinguishable region, e.g., a tumor or an inflammation, is present in the body section.

An elevated temperature or non-uniform temperature or a non-uniform temperature pattern may be generally associated with a tumor due to the metabolic abnormality of the tumor and proliferation of blood vessels (angiogenesis) at and/or near the tumor and on the breast surface. In a cancerous tumor the cells may double faster and thus may be more active and generate more heat. This tends to enhance the temperature differential between the tumor itself and the surrounding temperature. The present embodiments may therefore be advantageously used for diagnosis of cancer, particularly, but not exclusively breast cancer.

The surface information used for the analysis may comprise spatial information as well as optionally thermal information.

The spatial information may comprise data pertaining to geometric properties of a non-planar (i.e. curved) surface which may at least partially enclose a three-dimensional volume. Generally, the non-planar surface may be a two-dimensional object embedded in a three-dimensional space. Formally, a non-planar surface may be a metric space induced by a smooth connected and compact Riemannian 2-manifold. Ideally, the geometric properties of the non-planar surface would be provided explicitly, for example, the slope and curvature (or even other spatial derivatives or combinations thereof) for every point of the non-planar surface. Yet, such information may be rarely attainable and the spatial information may be provided for a sampled version of the non-planar surface, which may be a set of points on the Riemannian 2-manifold and which may be sufficient for describing the topology of the 2-manifold. Typically, the spatial information of the non-planar surface may be a reduced version of a three-dimensional spatial representation, which may be either a point-cloud or a three-dimensional reconstruction (e.g., a polygonal mesh or a curvilinear mesh) based on the point cloud. The three-dimensional spatial representation may be expressed via a three-dimensional coordinate system, such as, but not limited to, Cartesian, Spherical, Ellipsoidal, three-dimensional Parabolic or Paraboloidal coordinate three-dimensional system.

The term “surface” is used herein as an abbreviation of the term “non-planar surface”.

The spatial data, in some embodiments of the present invention, may be in a form of an image. Since the spatial data may represent the surface, such image is typically a two-dimensional image which, in addition to indicating the lateral extent of body members, may further indicate the relative or absolute distance of the body members, or portions thereof, from some reference point, such as the location of the imaging device. Thus, the image may typically include information residing on a non-planar surface of a three-dimensional body and not necessarily in the bulk. Yet, it is commonly acceptable to refer to such image as “a three-dimensional image” because the non-planar surface is conveniently defined over a three-dimensional system of coordinate. Thus, throughout this specification and in the claims section that follows, the terms “three-dimensional image” and “three-dimensional representation” primarily relate to surface entities.

The thermal information may comprise data pertaining to heat evacuated from or absorbed by the surface and/or to an IR (Infra Red) radiation emitted from the surface. Since different parts of the surface may generally evacuate or absorb different amount of heat, the thermal information may comprise a set of tuples, each may comprise the coordinates of a region or a point on the surface and a thermal numerical value (e.g., temperature, thermal energy) associated with the point or region. The thermal information may be transformed to visible signals, in which case the thermal information may be in the form of a thermographic image. The terms “thermographic image”, “IR image”, “thermal image” and “thermal information” are used interchangeably throughout the specification without limiting the scope of the present invention in any way. Specifically, unless otherwise defined, the use of the term “thermographic image” is not to be considered as limited to the transformation of the thermal information into visible signals. For example, a thermographic image may be stored in the memory of a computer readable medium as a set of tuples as described above.

The surface information (thermal and spatial) of a body may be typically in the form of a synthesized representation which may include both IR data representing the IR image and spatial data representing the surface, where the IR data may be associated with the spatial data (i.e., a tuple of the spatial data is associated with a heat-related value of the IR data). Such representation may be referred to as an IR-spatial representation. The IR-spatial representation may be in the form of digital data (e.g., a list of tuples associated with digital data describing thermal quantities) or in the form of an image (e.g., a three-dimensional image color-coded or grey-level coded according to the IR data). An IR-spatial representation in the form of an image is referred to hereinafter as an IR-spatial image.

The IR-spatial image may be defined over a three-dimensional spatial representation of the body and has thermal data associated with a surface of the three-dimensional spatial representation, and arranged gridwise over the surface in a plurality of picture-elements (e.g., pixels, arrangements of pixels), each represented by an intensity value or a grey-level over the grid. It is appreciated that the number of different intensity value may be different from the number of grey-levels. For example, an 8-bit display may generate 256 different grey-levels. However, in principle, the number of different intensity values corresponding to thermal information may be much larger. As a representative example, suppose that the thermal information spans over a range of 37° C. and may be digitized with a resolution of 0.1° C. In this case, there may be 370 different intensity values and the use of grey-levels may be less accurate by a factor of approximately 1.4. In some embodiments of the present invention the processing of thermal data may be performed using intensity values, temperature values, and in some embodiments of the present invention the processing of thermal data may be performed using grey-levels. Combinations of the two (such as double processing) may be also contemplated.

The term “pixel” is sometimes abbreviated herein to indicate a picture-element. However, this is not intended to limit the meaning of the term “picture-element” which refers to a unit of the composition of an image.

When the IR-spatial representation may be in the form of digital data, the digital data describing thermal properties may also be expressed either in terms of intensities or in terms of grey-levels as described above. Digital IR-spatial representation may also correspond to IR-spatial image whereby each tuple corresponds to a picture-element of the image.

Typically, one or more IR images, either measured or calculated, may be mapped onto the surface of the three-dimensional spatial representation to form the IR-spatial representation. The IR image to be mapped onto the surface of the three-dimensional spatial representation may comprise thermal data and/or IR data which may be expressed over the same coordinate system as the three-dimensional spatial representation. Any type of thermal data may be used. In one embodiment the thermal data may comprise absolute temperature values. In another embodiment the thermal data may comprise relative temperature values, each corresponding to, e.g., a temperature difference between a respective point of the surface and some reference point. In an additional embodiment, the thermal data may comprise local temperature differences. Also contemplated, are combinations of the above types of temperature data, for example, the thermal data may comprise both absolute and relative temperature values, and the like.

Typically, but not obligatorily, the information in the thermographic image may also include the thermal conditions (e.g., temperature) at one or more reference markers.

The mapping of the thermographic image onto the surface of the three-dimensional spatial representation may be done by positioning the reference markers, (e.g., by comparing their coordinates in the IR image with their coordinates in the three-dimensional spatial representation), to thereby match also other points hence to form the synthesized IR-spatial representation.

Optionally, the mapping of IR images may be accompanied by a correction procedure in which thermal emissivity considerations may be employed.

The thermal emissivity of a body member is a dimensionless quantity defined as the ratio between the amount of IR radiation emitted from the surface of the body member and the amount of IR radiation emitted from a black body having the same temperature as the body member. Thus, the thermal emissivity of an idealized black body is 1 and the thermal emissivity of all other bodies is between 0 and 1. It is commonly assumed that the thermal emissivity of a body is generally equal to its thermal absorption factor.

The correction procedure may be performed using estimated thermal characteristics of the body of interest. Specifically, the IR image may be mapped onto a non-planar surface describing the body taking into account differences in the emissivity of regions on the surface of the body and the emissivity's angular dependence. A region with a different emissivity value compared to its surroundings may be, for example, a scarred region, a pigmented region, a nipple region on the breast, a nevus, etc. In addition, assuming that the human skin is not perfect Lambertian source, the emissivity is angle dependent. Another consideration should take into account the possibility that the emissivity values of subjects with different skin colors may differ.

In some embodiments of the present invention, the IR image may be weighted according to the different emissivity values of the surface. For example, when information acquired by an IR imaging device include temperature or energy values, at least a portion of the temperature or energy values may be divided by the emissivity values of the respective regions on the surface of the body. One of ordinary skill in the art may appreciate that such procedure results in effective temperature or energy values which might be different than the values acquired by the IR imaging device. Since different regions may be characterized by different emissivity values, the weighted IR image may provide better estimation regarding the heat emitted from the surface of the body.

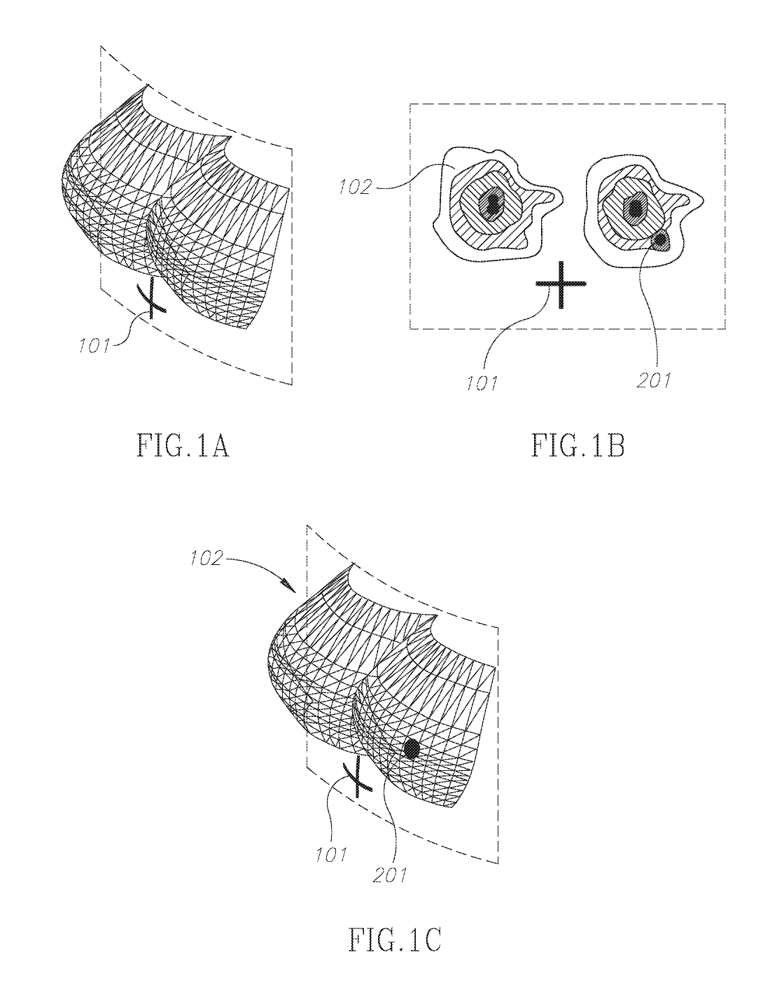

A representative example of a synthesized IR-spatial image for the case that the body comprise the breasts of a woman is illustrated in FIGS. 1A-C, which show a three-dimensional spatial representation illustrated as a non-planar surface (FIG. 1A), a thermographic image illustrated as planar isothermal contours (FIG. 1B), and a synthesized IR-spatial image formed by mapping the thermographic image on a surface of the three-dimensional spatial representation (FIG. 1C). As illustrated, the IR data of the IR-spatial image may be represented as grey-level values optionally but not necessarily over a grid generally shown at 102. It is to be understood that the representation according to grey-level values is for illustrative purposes and is not to be considered as limiting. As explained above, the processing of thermal data may also be performed using intensity values. Also shown in FIGS. 1A-C, is a reference marker 101 which optionally, but not obligatorily, may be used for the mapping.

The three-dimensional spatial representation, thermographic image and synthesized IR-spatial image may be obtained in any technique known in the art, such as the technique disclosed in International Patent Publication No. WO 2006/003658, U.S. Published Application No. 20010046316, and U.S. Pat. Nos. 6,442,419, 6,765,607, 6,965,690, 6,701,081, 6,801,257, 6,201,541, 6,167,151, 6,167,151, 6,094,198 and 7,292,719.

Some embodiments of the invention may be embodied on a tangible medium such as a computer (or “hardware data processor”) for performing the method steps. Some embodiments of the invention may be embodied on a computer readable medium, comprising computer readable instructions for carrying out the method steps. Some embodiments of the invention may also be embodied in electronic device having digital computer capabilities arranged to run the computer program on the tangible medium or execute the instruction on a computer readable medium. Computer programs implementing method steps of the present embodiments may commonly be distributed to users on a tangible distribution medium. From the distribution medium, the computer programs may be copied to a hard disk or a similar intermediate storage medium. The computer programs may be run by loading the computer instructions either from their distribution medium or their intermediate storage medium into the execution memory of the computer, configuring the computer to act in accordance with the method of this invention. All of these operations are well-known to those skilled in the art of computer systems.

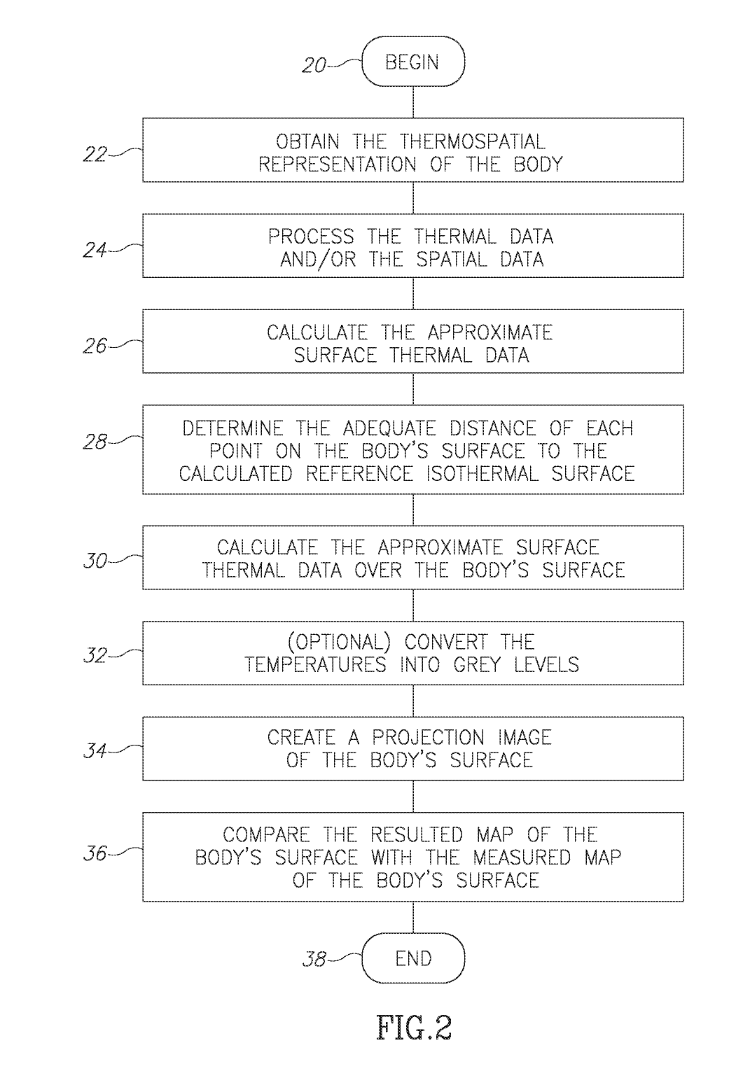

FIG. 2 shows a flow chart of a method suitable for analyzing a thermal image of a body section, according to some embodiments of the present invention. It is to be understood that several method steps appearing in the following description or in the flowchart diagram of FIG. 2 are optional and may not be executed.

The method may begin at step 20 and may continue to step 22 in which a spatial thermal representation (also referred as an IR-spatial representation) of the curved body section is obtained. The IR-spatial representation, as stated, may include IR data representing the thermal image and spatial data representing a non-planar surface of the curved body section, where the IR data may be associated with spatial data. The IR-spatial representation may be generated the method or it may be generated by another method or system from which the IR-spatial representation may be read by the method.

Optionally, the method may continue to step 24 in which the data in the IR-spatial representation may be preprocessed. The preprocessing may be done for the thermal data, the spatial data, or the both spatial and IR data.

Preprocessing of IR data may include, without limitation, powering (e.g., squaring), normalizing, enhancing, smoothing and the like. Preprocessing of spatial data may include, without limitation, removal, replacement and interpolation of picture-elements, using various processing operations such as, but not limited to, morphological operations (e.g., erosion, dilation, opening, closing), resizing operations (expanding, shrinking), padding operations, equalization operations (e.g., via cumulative density equalization, histogram equalization) and edge detection (e.g., gradient edge detection).

The method may proceed to step 26 which may be the first step for calculating the theoretical thermal simulation over the surface in an analytically method or in any other known method. There may be two major ways for calculating the temperature of external body surface; solving analytically the heat transfer equation with the proper boundary conditions and numerically by finite-element calculations or by other numerical calculations techniques. Analytical heat transfer equation solutions exist only for plane surfaces or symmetrical bodies like sphere or cylinder. For nonsymmetrical bodies the finite-element method should be applied. However, the finite-element method is may be too complicated when working in real time or with large shapes with variety shapes and boundary conditions. Thus, different approach may be adopted. In the present approach the theoretical thermal simulation over the surface may be calculated analytically based on known analytical heat transfer equation solutions (also referred as predetermined thermodynamic logic) based on behavior of a normal healthy body and the spatial data representing a non-planar surface of the curved body section. The first step in the calculation may be to define a reference point or isothermal surface in the body.

Once the reference isothermal surface in the body may be defined, the method may continue to step 28 in which the adequate distance of each point on the body's surface to the calculated reference isothermal surface may be determined. In general, the adequate distance of each point on the body's surface to the calculated reference isothermal surface may be simply the distance between the point on the body's surface to the nearest point on the calculated reference isothermal surface. The adequate distance may also be determined by any other function. It may also be improved based on trial and error Finite Element software (e.g. ANSYS) calculations.

After the adequate distance of each point on the body's surface to the calculated reference isothermal surface may be determined, the method may continue to step 30 in which the theoretical thermal simulation and/or IR data over the surface may be calculated. In general, but not limited to, the calculation of a body thermal map may be based on predetermined thermodynamic logic, for example the Pennes's bio-heat equation. The solution of the Pennes's bio-heat equation with the proper boundary conditions may determine the temperature of each point in the body as a function of its coordinates.

For example, the Pennes's bio-heat equation of the human's body in cylindrical coordinates is:

$$\frac{1}{r} \frac{d}{d r} ( r \frac{d T}{d r} ) - \frac{{W}_{b} {C}_{b}}{{K}_{t}} ( T - {T}_{art} ) = 0$$

Where:

W_{b}—is the volumetric blood perfusion rate (kg/s m3)

C_{b}—is blood specific heat (J/kg ° C.)

Kₜ—is tissue thermal conductivity (W/m K)

Tart—is arterial blood temperature (° C.)

r—Radius (m)

Solving this differential equation for certain boundary conditions may attain an equation that may give the temperature as a function of r—the distance of a point from the cylinder axis.

Since the Pennes's bio-heat equation may be applicable only for symmetrical bodies, in many researches the human body thermal behavior was calculated using solutions of the Pennes's bio-heat equation when parts of the human body's surface were approximated by cylinders. In these researches, it has been found that the surface thermal data over the body's surface calculated based on Pennes bio-heat equation are comparable to the measured surface thermal data with compatibility of higher than 95%. In order to increase the surface thermal data accuracy's calculations, the present method may obtain the theoretical thermal simulation by combining the actual spatial data of the human body's surface and known predetermined thermodynamic logic (the analytical solutions of the Pennes's bio-heat equation for symmetrical bodies in the example herein). In these approximations, the temperature of each point on the body's surface may be calculated by considering its spatial coordinates relative to the reference isothermal surface as the actual spatial coordinates and setting them in the Pennes's bio-heat equation's solution. For example, when solving the Pennes's bio-heat equation of the human's body in cylindrical coordinates, the appropriate distance of each point on the body's surface to the reference isothermal surface is considered as r and by setting it in the solution, the temperature at each point may be calculated. This method may also be used for calculating approximately the temperature inside the body by considering the spatial coordinates of each point as r, setting it in the solution and calculating the temperature at that point. Accordingly, other analytical solutions of the Pennes's bio-heat equation for other boundary conditions may be used for surface thermal data calculations, such as an analytical solution for half sphere. Using this solution, a half sphere may be fitted to the body's surface by least square techniques and the temperature at each point of the body's surface may be defined as the analytical calculated temperature at a suitable point with the same coordinated inside the half sphere. In another embodiment, the analytical solutions of the Pennes's bio-heat equation for ellipsoidal boundary conditions may be used for surface thermal data calculations. Using this solution, a proper half ellipsoid may be fitted to the body's surface by least square techniques and the temperature at each point of the body's surface may be defined as the analytical calculated temperature at a suitable point with the same coordinated inside the half ellipsoid. This method may also be used for calculating approximately the temperature inside the body by setting the spatial coordinates of each point in the solution and calculating the temperature at that point. A proper half ellipsoid may also be determined by the user. By marking several points on the body's surface, automatic software may tit the best fitted half ellipsoid to body's surface.

After calculating the temperature map at each point of the body's surface the method may continue to step 32 in which the temperature may be converted into grey levels. The conversion scale may be based on a calibration target.

The next step 34 may match the calculated temperature map to the 3D model, for example by creating a projection image of the body's surface to create the theoretical thermal simulation (i.e. simulate the scene viewed by a thermal camera). In this stage a correction procedure may be performed using estimated thermal characteristics of the body of interest. Specifically, the emissivity's angular dependence may be taken into account.

The next step 36 may compare the resulted theoretical thermal simulation of the body's surface with the thermal image of the body's surface obtained by the IR camera. By this comparison, a decision may be made whether or not the curved body section has an abnormality and/or pathology such as a tumor.

The method may end at step 38.

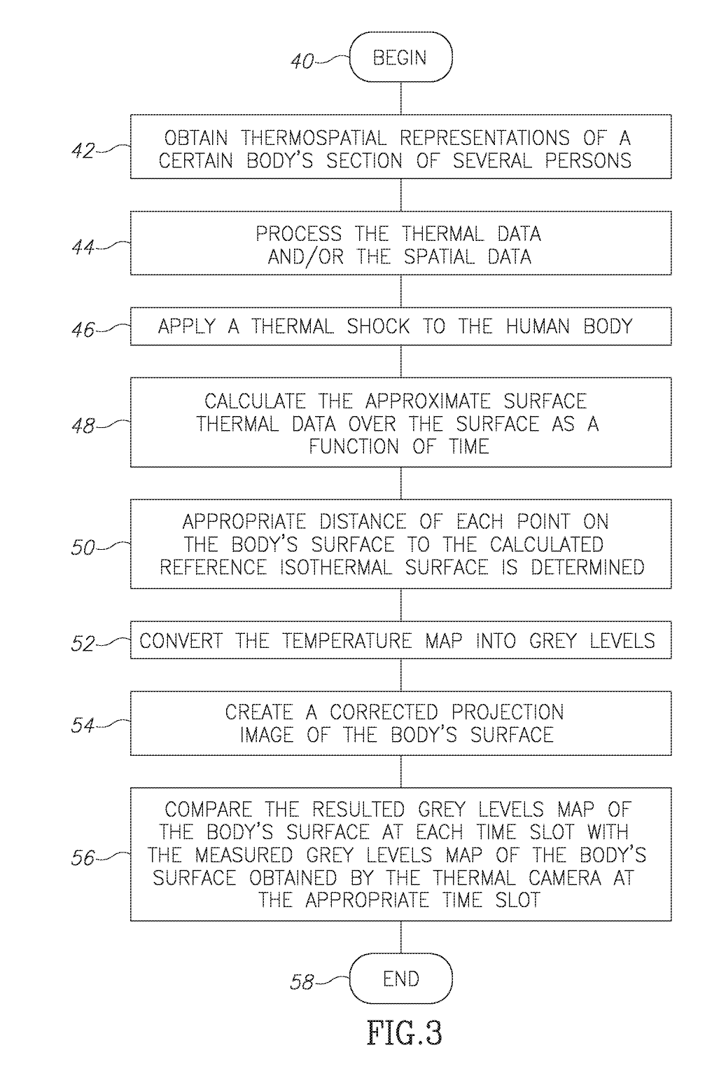

FIG. 3 shows a flowchart of another method suitable for analyzing a thermal image of a curved body section, according to some embodiments of the present invention. It is to be understood that several method steps appearing in the following description or in the flowchart diagram of FIG. 3 are optional and may not be executed.

The method may begin at step 40 and continue to step 42 in which a spatial thermal representation (also referred as an IR-spatial representation) of the curved body section may be obtained. The IR-spatial representation, as stated, may include IR data representing the thermal image and spatial data representing a non-planar surface of the curved body section, where the thermal data may be associated with spatial data. This IR-spatial representation may serve as initial boundary conditions for later calculations. The IR-spatial representation may be generated by the method or it may be generated by another method or system from which the IR-spatial representation may be read by the method.

Optionally, the method may continue to step 44 in which the data in the IR-spatial representation may be preprocessed. The preprocessing may be done for the thermal data, the spatial data, or the both spatial and thermal data.

Preprocessing of thermal data may include, without limitation, powering (e.g., squaring), normalizing, enhancing, smoothing and the like. Preprocessing of spatial data may include, without limitation, removal, replacement and interpolation of picture-elements, using various processing operations such as, but not limited to, morphological operations (e.g., erosion, dilation, opening, closing), resizing operations (expanding, shrinking), padding operations, equalization operations (e.g., via cumulative density equalization, histogram equalization) and edge detection (e.g., gradient edge detection).

The method may continue to step 46 in which a thermal shock may be applied to the human body.

The method may continue to step 48 which may be the first step for analytically calculating the theoretical thermal simulation over the surface as a function of time. As mentioned above, there are two major ways for calculating the temperature of external body surface as a function of time; solving analytically the time dependent partial differential heat transfer equation with the proper boundary conditions or numerically by FDTD (Finite Differences Time Domain) calculations or other numerical techniques. Analytical solutions for the heat transfer time dependent equation may exist only for plane surfaces or symmetrical bodies like sphere or cylinder. For nonsymmetrical bodies the FDTD methods should be applied. In the present approach the theoretical thermal simulation over the surface may be calculated analytically based on known analytical heat transfer time dependent equation solutions (also referred as predetermined thermodynamic logic) based on behavior of a normal healthy body, the initial thermal data and the spatial data representing a non-planar surface of the curved body section. The first step in the calculation may be to define a reference isothermal surface in the body. The reference isothermal surface in the body may be obtained by virtually “removing” the actual breasts from the spatial data representing the non-planar surface of the body section and extrapolating the surface at the vacancies using the surrounding spatial data. The reference isothermal surface in the body may also be obtained by approximating the surface at the vacancies with a planar surface or any other non-planar surface. The adequate non-planar surface definition may also be improved based on trial and error Finite Element software (e.g. ANSYS) IR-spatial calculations.

Once the reference isothermal surface in the body is defined, the method may continue to step 50 in which the adequate distance of each point on the body's surface to the calculated reference isothermal surface may be determined. In general, the appropriate distance of each point on the body's surface to the calculated reference isothermal surface may be simply the distance between the point on the body's surface to the nearest point on the calculated reference isothermal surface. The appropriate distance may also be determined by any other function. It may also be improved based on trial and error Finite Element software (e.g. ANSYS) calculations.

After the adequate distance of each point on the body's surface to the calculated reference isothermal surface is determined, the theoretical thermal simulation over the surface as a function of time may be calculated. In general, the calculation of a body thermal map as a function of time may be based on predetermined thermodynamic logic, for example the partial differential heat transfer equation with the proper human tissue and blood thermal parameters under convective and radiative boundary conditions. The solution of said partial differential heat transfer equation may determine the connection between the spatial coordinates of a point in the body and its temperature as a function of time.

Since solution for said partial differential heat transfer equation may be applicable only for simple bodies the present method may obtain the time dependent theoretical thermal simulation by combining the actual spatial data of the human body's surface and known predetermined thermodynamic logic (the analytical solutions of said partial differential heat transfer equation in the example herein). In these approximations, the temperature of each point on the body's surface at a certain time may be calculated by considering its spatial coordinates relative to the reference isothermal surface as the actual spatial coordinates and setting them and the time in a solution of said partial differential heat transfer equation. As for example, the partial differential heat transfer equation for a plane with thickness L, under convective and radiative boundary conditions and with initial temperature boundary conditions may be solved analytically. The measured temperature at each surface point may be considered as the initial temperature for the boundary conditions. The appropriate distance of each point on the body's surface to the reference isothermal surface may be considered as L. Setting it, the appropriate initial temperatures and the time in the solution, the temperature at each point as a function of time may be calculated. Accordingly, other analytical solutions of the partial differential heat transfer equation for other geometrical bodies may be used for surface thermal data calculations, such as an analytical solution for half sphere. Using this solution, a half sphere may be fitted to the body's surface by least square techniques and the temperature at each point of the body's surface may be defined as the analytical calculated temperature at a suitable point with the same coordinated inside the half sphere as a function of time when the measured temperature at each surface point may be considered as the initial temperature for the boundary conditions. In another scheme the partial differential heat transfer equation may be solved for half sphere with radius L, under convective and radiative boundary conditions and initial temperatures boundary conditions, the appropriate distance of each point on the body's surface to the reference isothermal surface may be considered as L and by setting it, the initial temperature and the time in the solution, the temperature at each point as a function of time may be calculated. In another embodiment, the analytical solutions of partial differential heat transfer equation for ellipsoidal body may be used for surface thermal data calculations. Using this solution, a proper half ellipsoid may be fitted to the body's surface by least square techniques and the temperature at each point of the body's surface as a function of time may be defined as the analytical calculated temperature as a function of time at a suitable point with the same coordinated inside the half ellipsoid when setting the initial conditions in the solution.

A proper half ellipsoid may also be determined by the user. By marking several points on the body's surface automatic software may fit the best fitted half ellipsoid to body's surface.

After calculating the temperature map at each point of the body's surface the method may continue to step 52 in which the temperature may be converted into grey levels. The conversion scale may be based on a calibration target.

The next step 54 may match the calculated temperature map to the 3D model, for example by creating a projection image of the body's surface to create the theoretical thermal simulation (i.e. simulate the scene viewed by a thermal camera). In this stage a correction procedure may be performed using estimated thermal characteristics of the body of interest. Specifically, the emissivity's angular dependence may be taken into account.

The next step 56 may compare the resulted theoretical thermal simulation (grey levels map) of the body's surface with the measured thermal image (grey levels map) of the body's surface obtained by the thermal camera. By this comparison a decision may be made whether or not the body section has an abnormality and/or pathology such as a tumor.

The method may end at step 58.

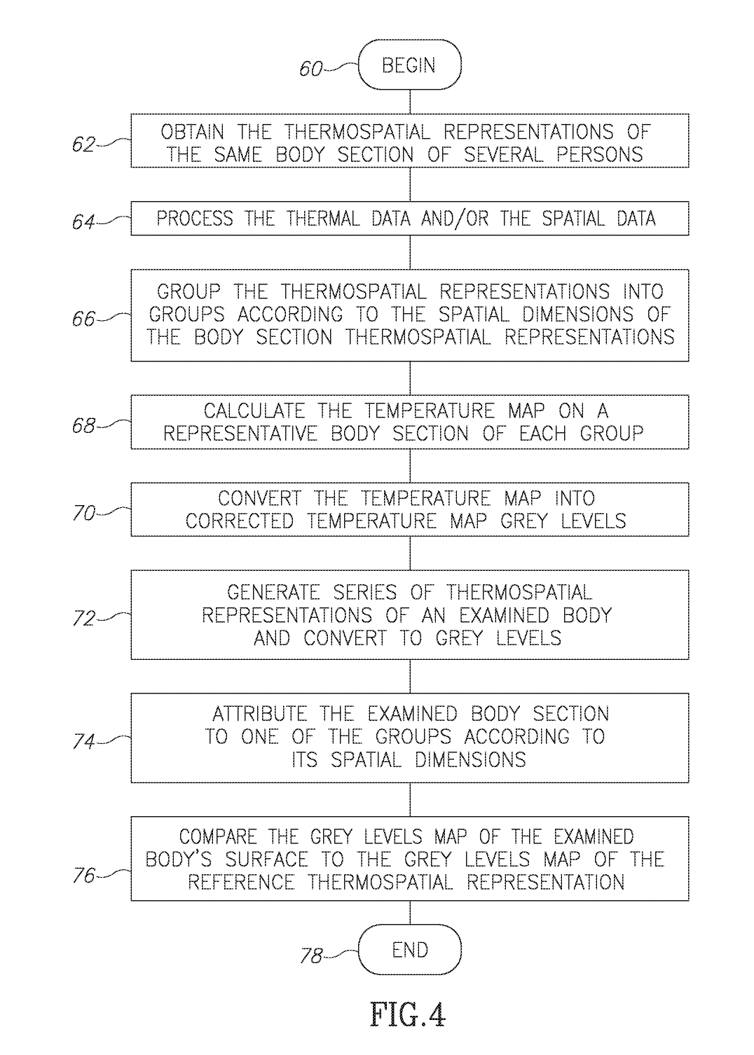

FIG. 4 shows a flowchart of another method suitable for analyzing a thermal image of a curved body section, according to some embodiments of the present invention. It is to be understood that several method steps appearing in the following description or in the flowchart diagram of FIG. 4 are optional and may not be executed.

The method may begin at step 60 and continue to step 62 in which a spatial thermal representations (also referred as IR-spatial representations) of the same curved body section of at least two persons are obtained.

Optionally, the method may continue to step 64 in which the data in the IR-spatial representation may be preprocessed. The preprocessing may be done for the thermal data, the spatial data, or the both spatial and thermal data.

The method may continue to step 66 in which said series IR-spatial representations of the at least two persons may be grouped into at least two groups according to the spatial characteristics of the curved body section. Each group may contain IR-spatial representations of body's sections with roughly the same spatial dimensions. The phrase “same spatial characteristics” means volume, or surface's area, or height, or length, or width, shape, etc.

The method may continue to step 68. In this step, for each group, all IR-spatial representations may be registered and morphed by deformation software into a representative body section. The temperature at each point at the representative body's surface may be calculated by averaging the thermal data at the corresponding point of all IR-spatial representations. The Obtained thermal image may be considered as a reference IR-spatial representation.

After calculating the temperature map of the reference IR-spatial representation the method may continue to step 70 in which the temperature may be optionally converted into grey levels. The conversion scale may be based on a calibration target. In this stage, a correction procedure may be performed to taken into account the emissivity's angular dependence of the surface.

Once the grey level reference IR-spatial representation of the body may be obtained, the method may continue to step 72 in which one or series of IR-spatial representations of an examined body section may be generated.

After generating a series of IR-spatial representations of an examined body section, the method may continue to step 74. In this stage the examined body section may be attributed to one of said groups according to its spatial characteristics. The body section may be then registered and morphed by deformation software into the representative body section of the present group.

The next step 76 may compare the resulted theoretical thermal simulation (grey levels map) of the examined body's surface with the measured thermal image (grey levels map) of the reference IR-spatial representation. By this comparison a decision may made whether or not the body section has an abnormality and/or pathology such as a tumor.

The method may end at step 78.

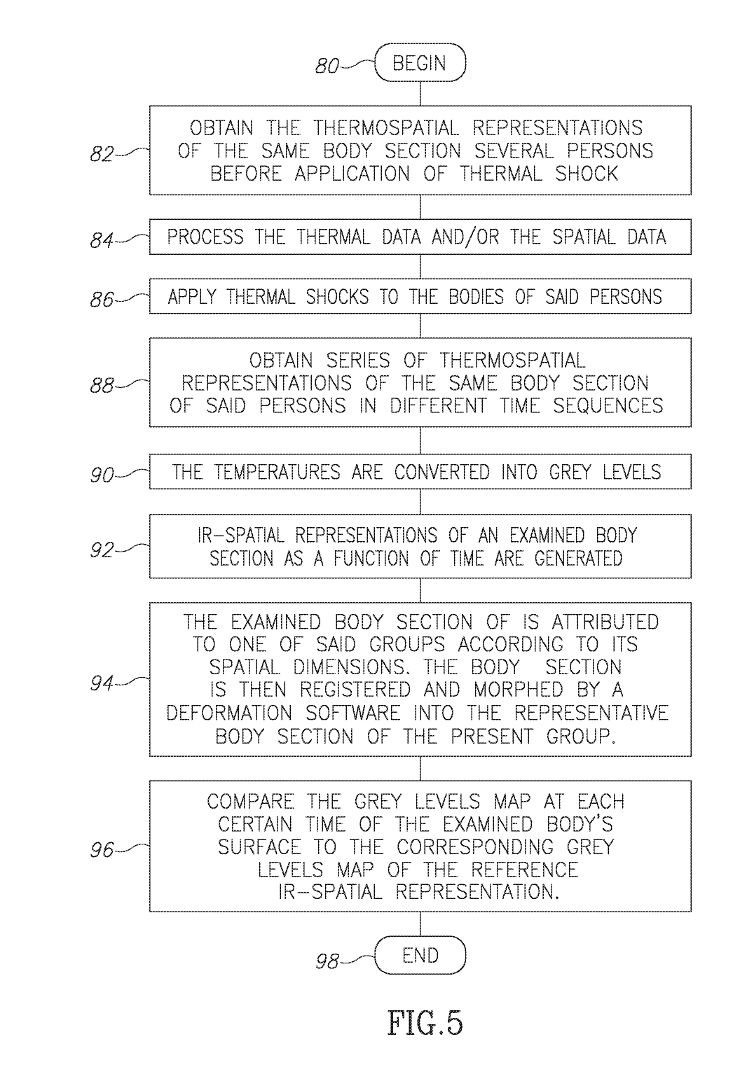

FIG. 5 shows a flowchart of another method suitable for analyzing a thermal image of a curved body section, according to some embodiments of the present invention.

The method may begin at step 80 and continue to step 82 in which a spatial thermal representations (also referred as IR-spatial representations) of the same curved body section of at least two persons after application of thermal shock may be obtained as a function of time.

Optionally, the method may continue to step 84 in which the data in the IR-spatial representation may be preprocessed. The preprocessing may be done for the thermal data, the spatial data, or the both spatial and thermal data.

The method may continue to step 86 in which said series IR-spatial representations as a function of time of the at least two persons are grouped into at least two groups according to the spatial dimensions of the body section. Each group may contain IR-spatial representations of body's sections with roughly the same spatial characteristics. The phrase “same spatial characteristics” means volume, surface's area, height, length, width, shape, etc.

The method may continue to step 88. In this step, for each group, all IR-spatial representations may be registered and morphed by deformation software into a representative body section. The temperature at each point at the representative body's surface as a function of time may be calculated by averaging the thermal data at the corresponding point and time of all IR-spatial representations. The obtained thermal images as a function of time may be considered as a reference IR-spatial representation;

After calculating the temperature maps of the reference IR-spatial representations as a function of time the method may continue to step 90 in which the temperatures may be converted into grey levels. The conversion scale may be based on a calibration target. In this stage a correction procedure may be performed to taken into account the emissivity's angular dependence of the surface.

Once the grey level reference IR-spatial representations of the body as a function of time may be obtained, the method may continue to step 92 in which series of IR-spatial representations of an examined body section as a function of time may be generated.

After generating a series of IR-spatial representations of an examined body section, the method may continue to step 94. In this stage, the examined body section may be attributed to one of said groups according to its spatial characteristics. The body section may then be registered and morphed by deformation software into the representative body section of the present group.

The next step 96 may compare the resulted theoretical thermal simulation (grey levels map) at each certain time of the examined body's surface with the corresponding measured thermal image (grey levels map) of the reference IR-spatial representation. By this comparison a decision is made whether or not the body section has an abnormality and/or pathology such as a tumor.

The method ends at step 98.

In all above-mentioned methods, there is more than one way to determine the likelihood for the presence of a thermally distinguishable region is the body section.

In some embodiments, the difference or the ratio of the reference grey levels map of the body's surface at different times and the measured grey levels map of the body's surface obtained by the thermal camera in different times may be compared to threshold values, and the comparison may be used for determining the likelihood for the presence of a thermally distinguishable region (also referred as an abnormality). Typically, but not obligatorily, when the difference or the ratio may be lower than the threshold, no thermally distinguishable region is present. The threshold values might be different for different times and different body sections.

In some embodiments, the imaging may be done in response to a cold stress test (a test in which, merely as an example, the subject holds a cold item, somehow changing blood flow in the body), in order to enhance the distinguish ability and thus improve the likelihood for distinguishing an abnormality.

Moreover, in some embodiments the location and/or size and/or shape of the abnormality (or thermally distinguishable region) inside the body may be estimated. For example, if the temperature of the thermally distinguishable region may be known, the region inside the body which has an approximate temperature that is comparable to the thermally distinguishable region's temperature may be estimated as the location of the thermally distinguishable region.

The reference grey levels map of the body's surface may be used as a platform for any kind of comparisons to the measured grey levels map of the body's surface obtained by the thermal camera. For example, a comparison of the integral of the grey levels values on the reference body's surface and the integral of the grey levels values on the measured body's surface. In another example, a comparison of the local standard deviation of the grey levels values on the reference body's surface and the local standard deviation of the grey levels values on the measured body's surface.

As delineated above, the calculation of the difference or the ratio of the resulted grey levels map of the reference body's surface at different times and the measured grey levels map of the body's surface obtained by the thermal camera in different times may be preceded by preprocessing operation.

In some embodiments of the present invention, the preprocessing operation may include a definition of a region-of-interest within the surface of the body section. In these embodiments, the difference or the ratio may be calculated over the region-of-interest. More than one region-of-interests may be defined, in which case the surface integral may be calculated separately for each region-of-interest. A region-of-interest may be defined, for example, as a part of the surface which is associated with high temperatures. A representative example of such region-of-interest may be a region surrounding a thermally distinguishable spot on the surface. FIG. 1C schematically illustrates a thermally distinguishable spot 201. The grey area surrounding spot 201 can be defined as a region-of-interest.

An IR-spatial representation or image may be generated obtained by acquiring one or more thermographic images and mapping the thermographic image(s) on a three-dimensional spatial representation.

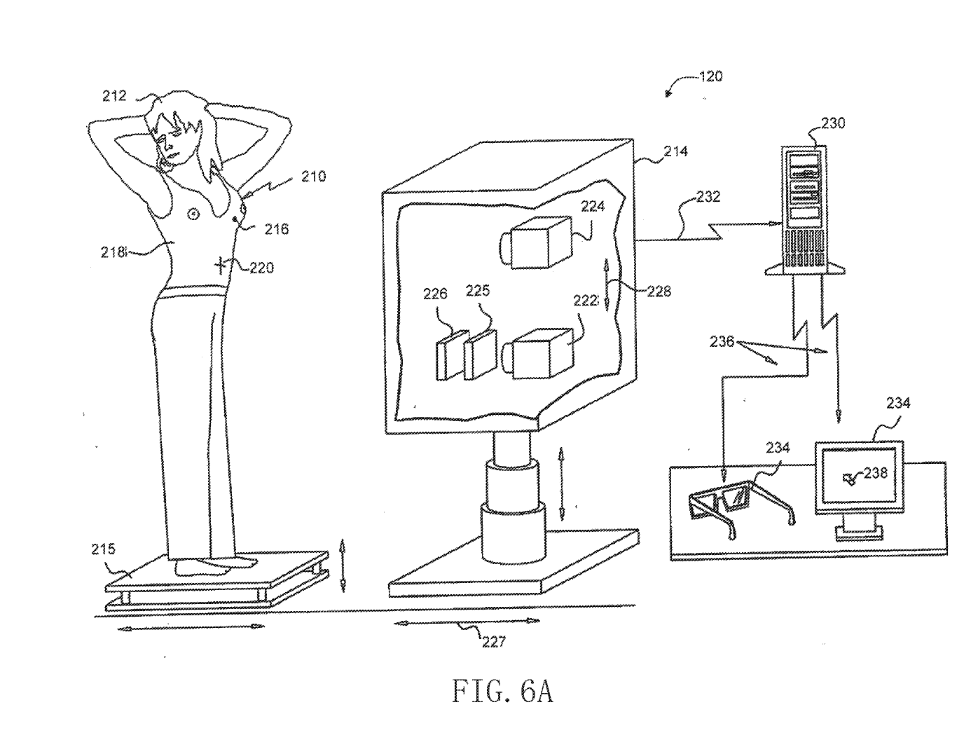

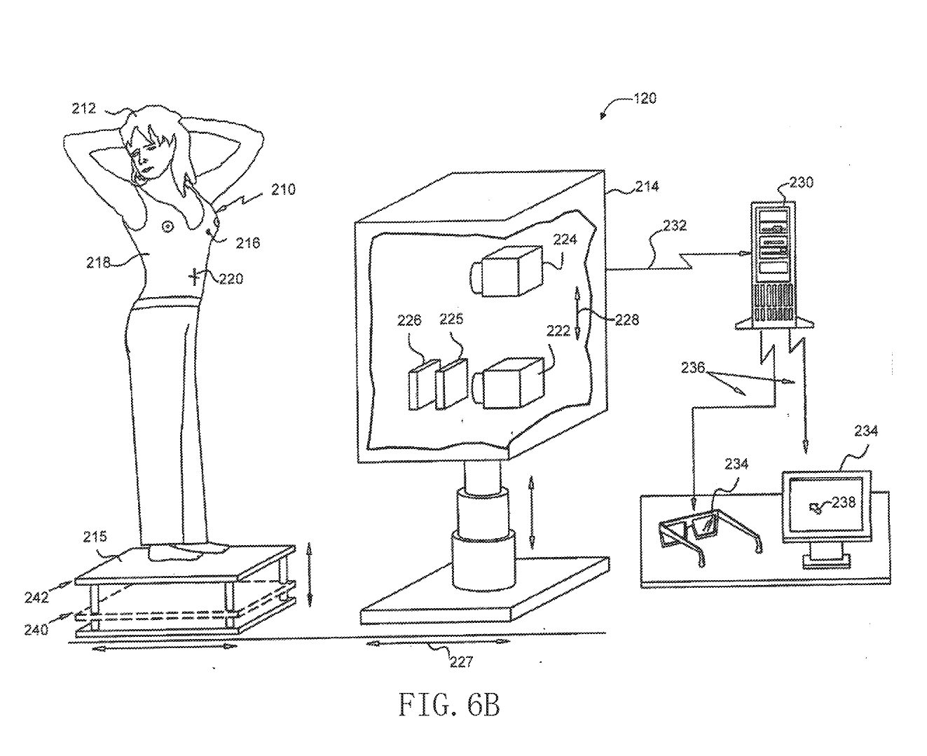

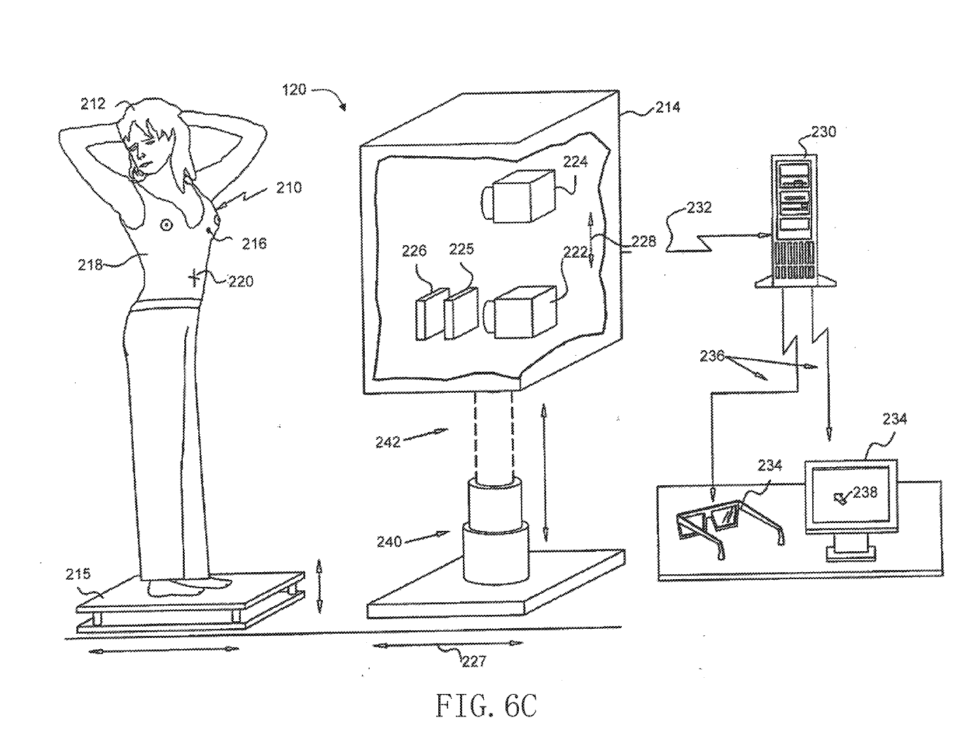

Reference is now made to FIG. 6A which shows a schematic illustration of an IR-spatial imaging system in accordance with embodiments of the present invention. An IR-spatial imaging system 120 is described. A living body 210 or a part thereof of a person 212 may be located in front of an imaging device 214. Person 212 may be standing, sitting or in any other suitable position relative to imaging device 214. Person 212 may initially be positioned or later be repositioned relative to imaging device 214 by a positioning device 215, which may typically comprise a platform moving on a rail, by force of an engine, or by any other suitable force. Additionally, a thermally distinguishable object 216, such as a tumor, may exist in body 210 of person 212. For example, when body 210 comprises a breast, object 216 may be a breast tumor such as a cancerous tumor.

In accordance with an embodiment of the present invention, person 212 may be wearing a clothing garment 218, such as a shirt. Clothing garment 218 may be non-penetrable or partially penetrable to visible wavelengths such as 400-700 nanometers, and may be penetrable to wavelengths that are longer than visible wavelengths, such as infrared wavelengths. Additionally, a reference mark 220 may be located close to person 212, optionally directly on the body of person 212 and in close proximity to body 210. Optionally, reference mark 220 may be directly attached to body 210. Reference mark 220 may typically comprise a piece of material, a mark drawn on person 212 or any other suitable mark, as described herein below.

Imaging device 214 may typically comprise at least one visible light imaging device 222 that may sense at least visible wavelengths and at least one thermographic imaging device 224 which may be sensitive to infrared wavelengths, typically in the range of as 3-5 micrometer and/or 8-12 micrometer. Typically imaging devices 222 and 224 may be capable of sensing reference mark 220 described hereinabove.

Optionally, a polarizer 225 may be placed in front of visible light imaging device 222. As a further alternative, a color filter 226, which may block at least a portion of the visible wavelengths, may be placed in front of visible light imaging device 222.

Typically, at least one visible light imaging device 222 may comprise a black-and-white or color stills imaging device, or a digital imaging device such as CCD or CMOS. Additionally, at least one visible light imaging device 222 may comprise a plurality of imaging elements, each of which may be a three-dimensional imaging element.

Optionally, imaging device 214 may be repositioned relative to person 212 by a positioning device 227. As a further alternative, each of imaging devices 222 and 224 may also be repositioned relative to person 212 by at least one positioning device 228. Positioning device 227 may comprise an engine, a lever or any other suitable force, and may also comprise a rail for moving imaging device 214 thereon. Repositioning device 228 may be similarly structured.

Data acquired by visible light imaging device 222 and thermographic imaging device 224 may be output to a data processor 230 via a communications network 232, and may be typically analyzed and processed by an algorithm running on the data processor. The resulting data may be displayed on at least one display device 234, which is optionally connected to data processor 230 via a communications network 236. Data processor 230 may typically comprise a PC, a PDA or any other suitable hardware data processor. Communications networks 232 and 236 may typically comprise a physical communications network such as an internet or intranet, or may alternatively comprise a wireless network such as a cellular network, infrared communication network, a radio frequency (RF) communications network, a blue-tooth (BT) communications network or any other suitable communications network.

In accordance with an embodiment of the present invention, display 234 typically comprises a screen, such as an LCD screen, a CRT screen or a plasma screen. As a further alternative display 234 may comprise at least one visualizing device comprising two LCDs or two CRTs, located in front of a user's eyes and packaged in a structure similar to that of eye-glasses. Display 234 may also display a pointer 238, which may be typically movable along the X, Y and Z axes of the displayed model and may be used to point to different locations or elements in the displayed data.

Reference is now made to FIGS. 6B-C and 7A-E which show illustrations of various operation principles of IR-spatial imaging system, in accordance with various exemplary embodiments of the invention.

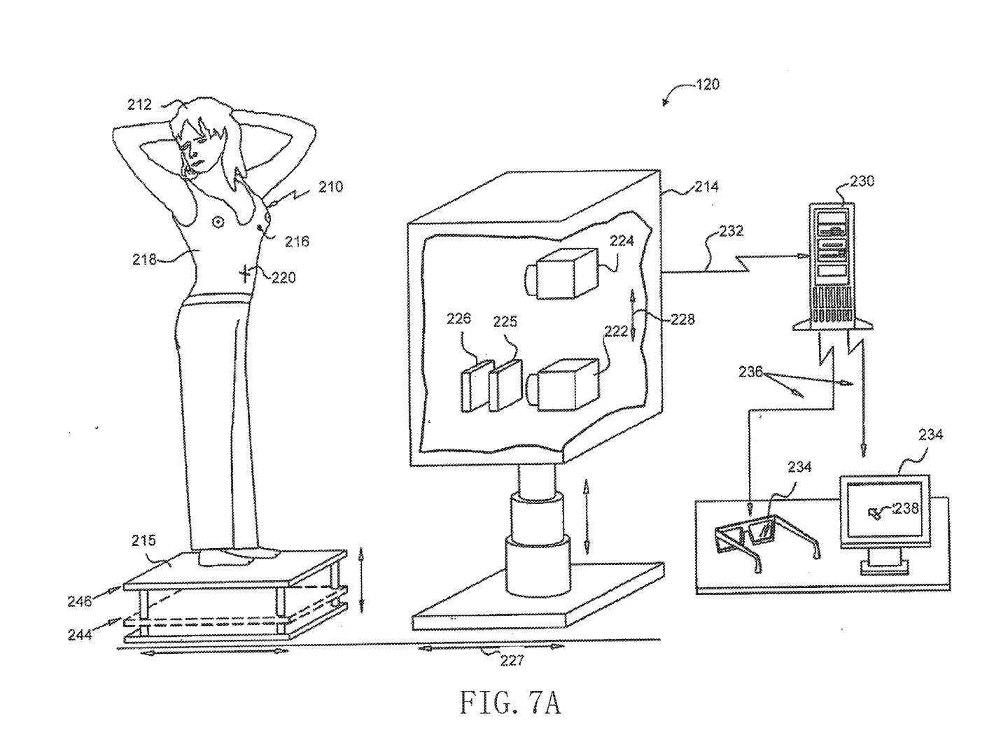

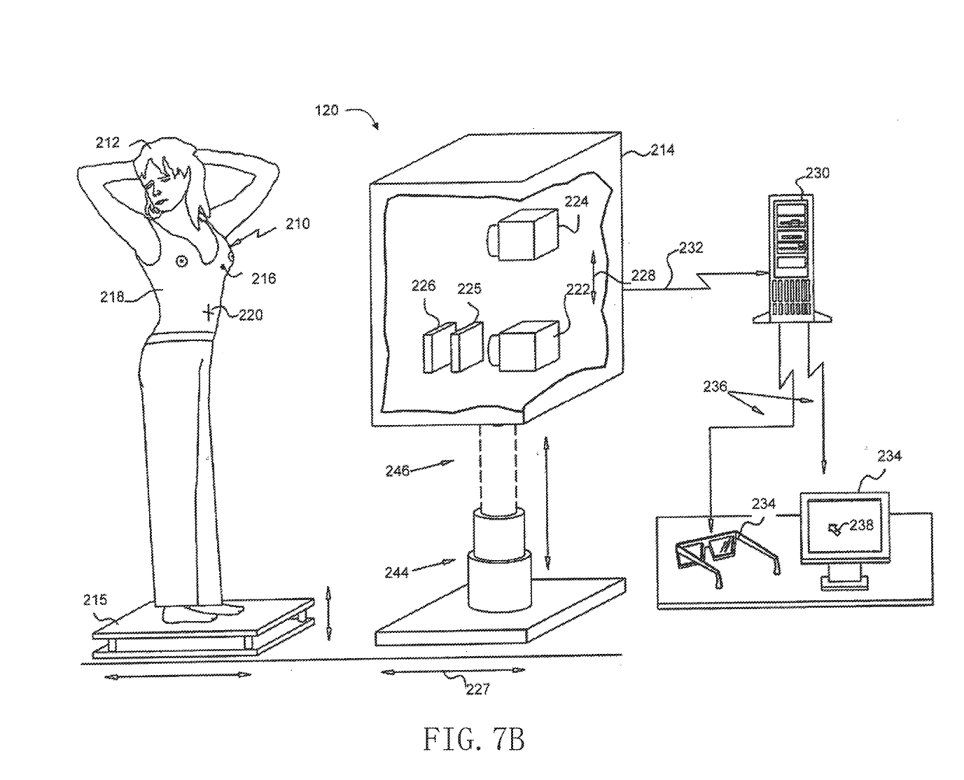

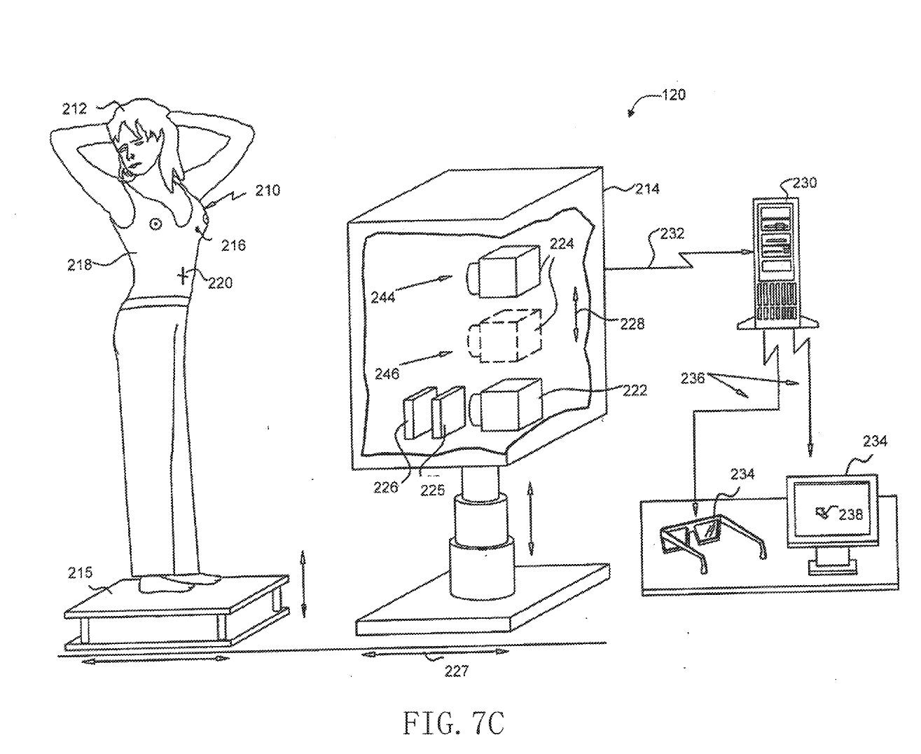

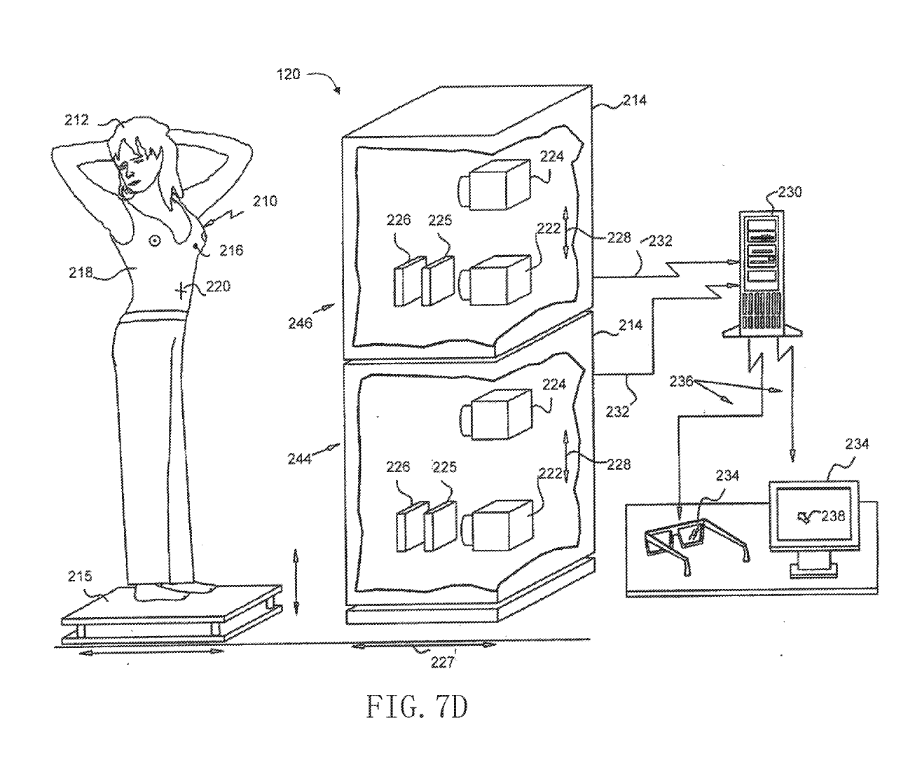

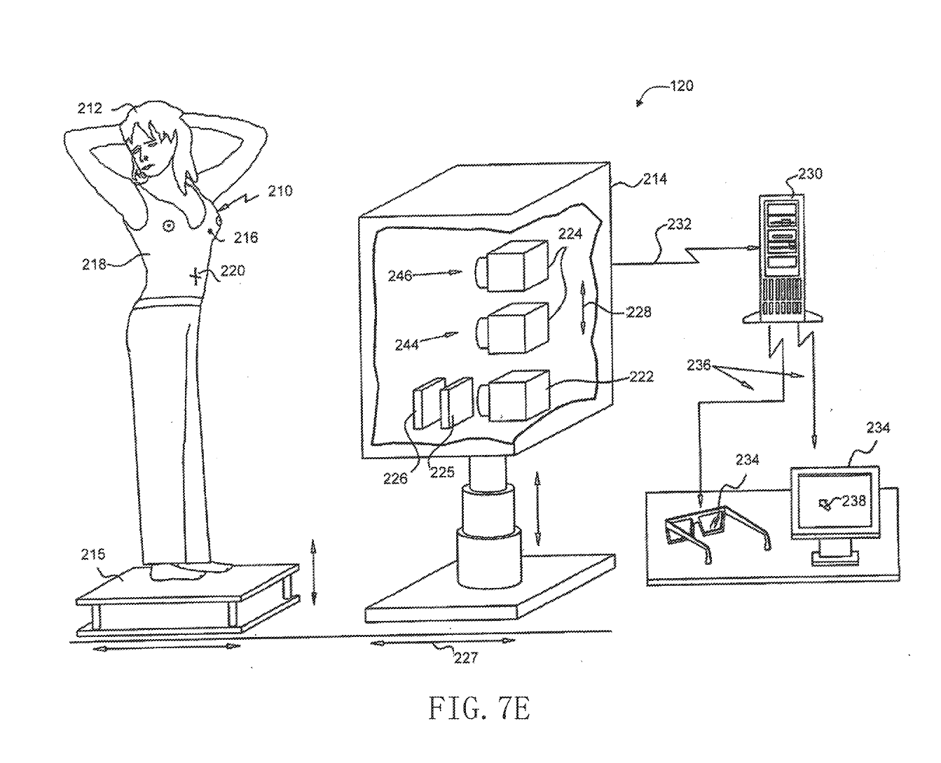

The visible light imaging is described first, with reference to FIGS. 6B-C, and the thermographic imaging is described hereinafter, with reference to FIGS. 7A-E. It will be appreciated that the visible light image data acquisition described in FIGS. 6B-C may be performed before, after or concurrently with the thermographic image data acquisition described in FIGS. 7A-E.

Referring to FIGS. 6B-C, person 212 comprising body 210 may be located on positioning device 215 in front of imaging device 214, in a first position 240 relative to the imaging device. First image data of body 210 may be acquired by visible light imaging device 222, optionally through polarizer 225 or as an alternative option through color filter 226. The advantage of using a color filter is that it may improve the signal-to-noise ratio, for example, when the person is illuminated with a pattern or mark of specific color, the color filter may be used to transmit only the specific color thereby reducing background readings. Additionally, at least second image data of body 210 is acquired by visible light imaging device 222, such that body 210 may be positioned in at least a second position 242 relative to imaging device 214. Thus, the first, second and optionally more image data may be acquired from at least two different viewpoints of the imaging device relative to body 210.

The second relative position 242 may be configured by repositioning person 212 using positioning device 215 as seen in FIG. 6B, by repositioning imaging device 214 using positioning device 227 as seen in FIG. 6C or by repositioning imaging device 222 using positioning device 228 as seen in FIG. 6C. As a further alternative, second relative position 242 may be configured by using two separate imaging devices 214 as seen in FIG. 7D or two separate visible light imaging device 222 as seen in FIG. 7E (with devices 224).

Referring to FIGS. 7A-E, person 212 comprising body 210 may be located on positioning device 215 in front of imaging device 214, in a first position 244 relative to the imaging device. First thermographic image data of body 210 may be acquired by thermographic imaging device 224. Optionally, at least second thermographic image data of body 210 may be acquired by thermographic imaging device 224, such that body 210 may be positioned in at least a second position 246 relative to imaging device 214. Thus, the first, second and optionally more thermographic image data may be acquired from at least two different viewpoints of the thermographic imaging device relative to body 210.

The second relative position 246 may be configured by repositioning person 212 using positioning device 215 as seen in FIG. 7A, by repositioning imaging device 214 using positioning device 227 as seen in FIG. 7B, or by repositioning thermographic imaging device 224 using positioning device 228 as seen in FIG. 7C. As a further alternative, second relative position 246 may be configured by using two separate imaging devices 214 as seen in FIG. 7D or two separate thermographic imaging devices 224 as seen in FIG. 7E.

Image data of body 210 may be acquired by thermographic imaging device 224, by separately imaging a plurality of narrow strips of the complete image of body 210. Alternatively, the complete image of body 210 may be acquired by the thermographic imaging device, and the image may be sampled in a plurality of narrow strips or otherwise shaped portions for processing. As a further alternative, the imaging of body 210 may be performed using different exposure times.

The thermographic and visible light image data obtained from imaging device 214 may be analyzed and processed by data processor 230 as follows. Image data acquired from imaging device 222 may be processed by data processor 230 to build a three-dimensional spatial representation of body 210, using algorithms and methods that are well known in the art, such as the method described in U.S. Pat. No. 6,442,419 which is hereby incorporated by reference as if fully set forth herein. The three-dimensional spatial representation may comprise the location of reference marker 220 (cf. FIG. 6A). Optionally, the three-dimensional spatial representation may comprise information relating to the color, hue and tissue texture of body 210. Thermographic image data acquired from imaging device 224 may be processed by data processor 230 to build a thermographic three-dimensional model of body 210, using algorithms and methods that are well known in the art, such as the method described in U.S. Pat. No. 6,442,419. The thermographic three-dimensional model may comprise reference marker 220 (cf. FIG. 7A). The thermographic three-dimensional model may then be mapped by processor 230 onto the three-dimensional spatial representation, e.g., by aligning reference marker 220, to form the IR-spatial image.

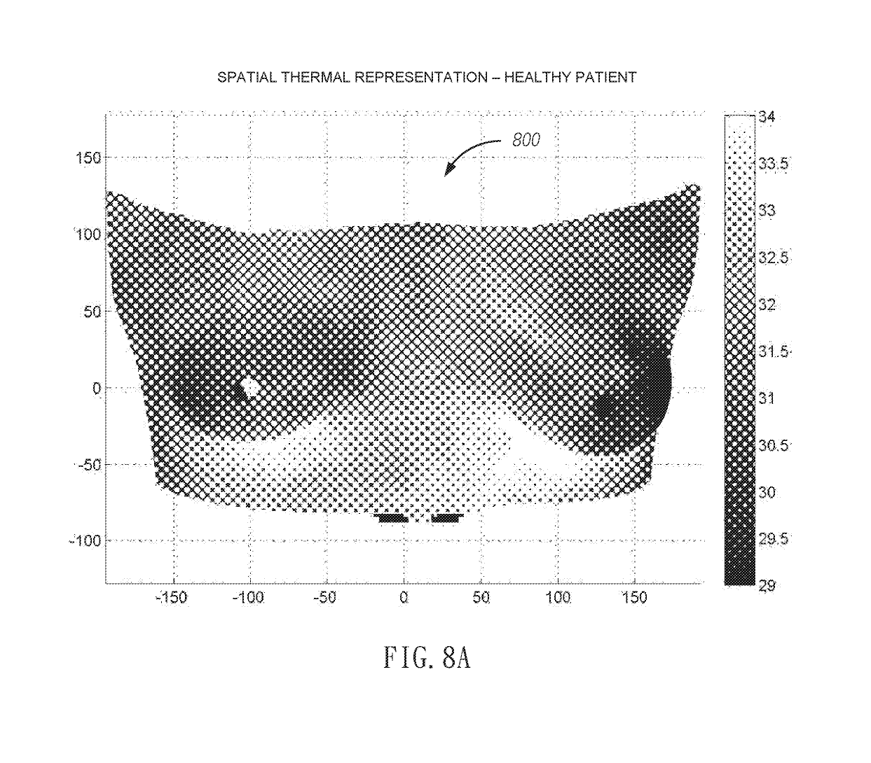

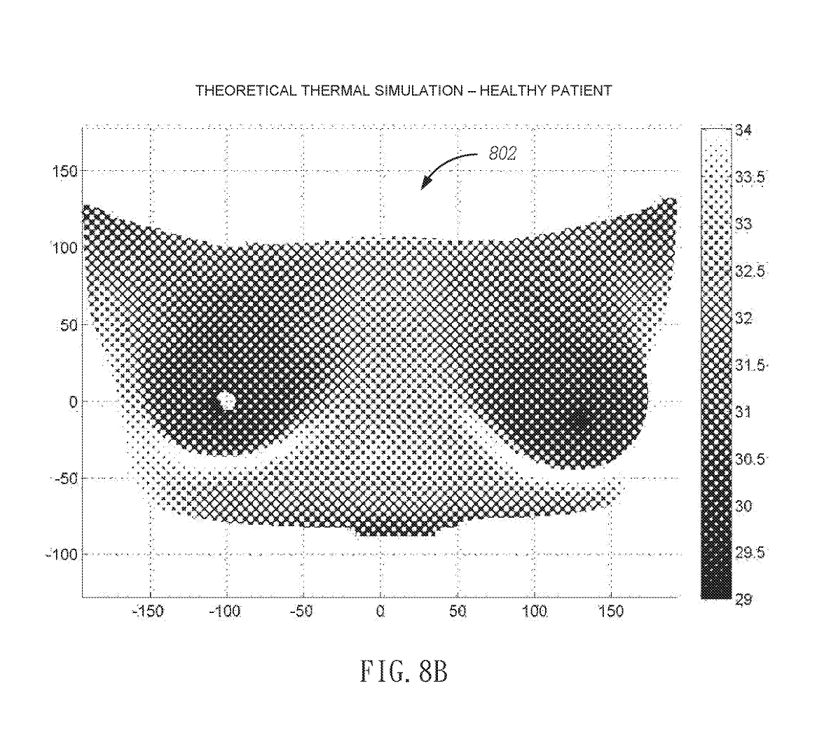

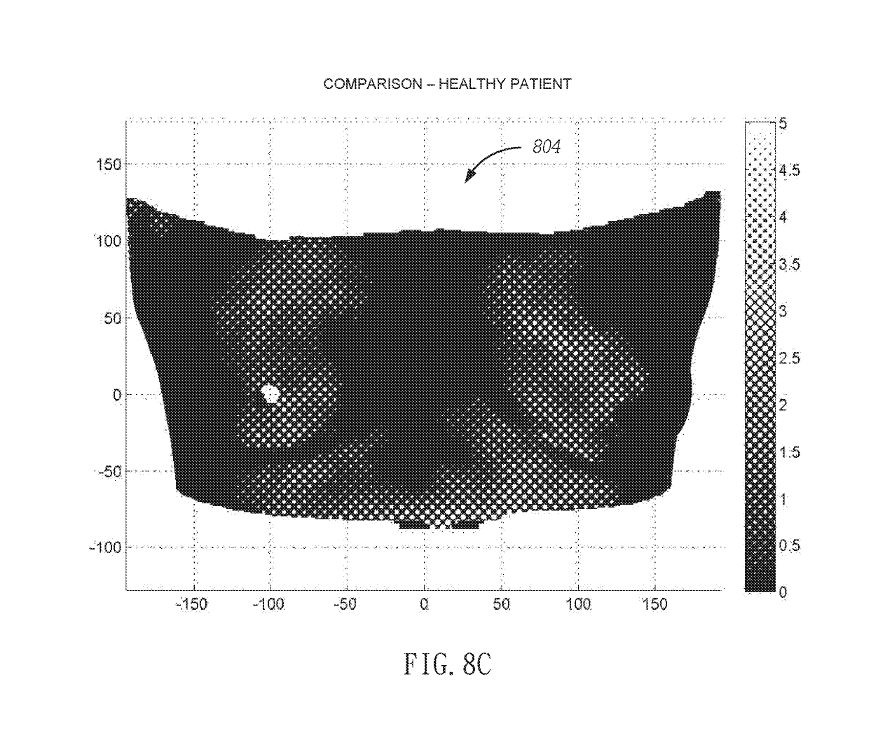

Reference is now made to FIGS. 8A, 8B and 8C, which show pictorial views of a spatial thermal representation 800, a theoretical thermal simulation 802 and a comparison 804, respectively—all of a healthy subject having no breast abnormalities (e.g. tumors). Representation 800 and simulation 802 are shown as a heat map, wherein darker areas mean lower temperature whereas lighter areas mean higher temperature. The heat map is displayed on a scale of 29 to 34 degrees Celsius.

As can be observed in FIG. 8A, spatial thermal representation 800 includes areas of different temperature which are randomly located, sized and shaped—as acquired in reality by the present imaging device. In contrast, theoretical thermal simulation 802 of FIG. 8B is shown with smoother and far more arranged temperature gradients. That is, theoretical thermal simulation 802 represents a mathematical model of temperature gradients of a 3D reconstruction of that patient's breasts.

Comparison 804 of FIG. 8C shows temperature differences between spatial thermal representation 800 and theoretical thermal simulation 802. As can be observed, the majority of the area of comparison 804 is indicative of a temperature difference of approximately 0 to 0.5 degrees Celsius, whereas the remaining area indicates a temperature difference of approximately 1-1.5 degrees Celsius. Namely, comparison 804 indicates that the temperature differences are relatively minimal.

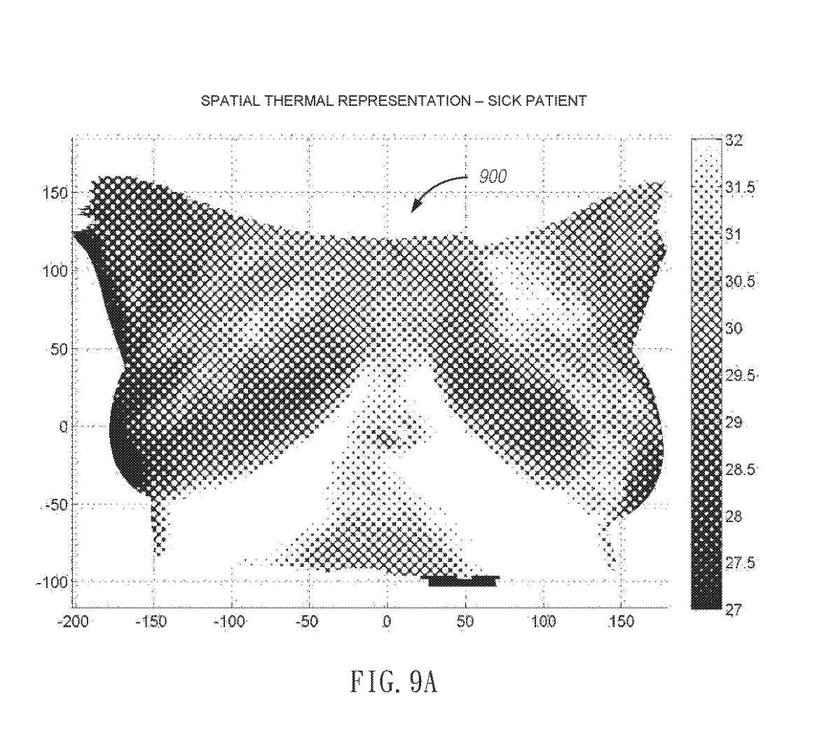

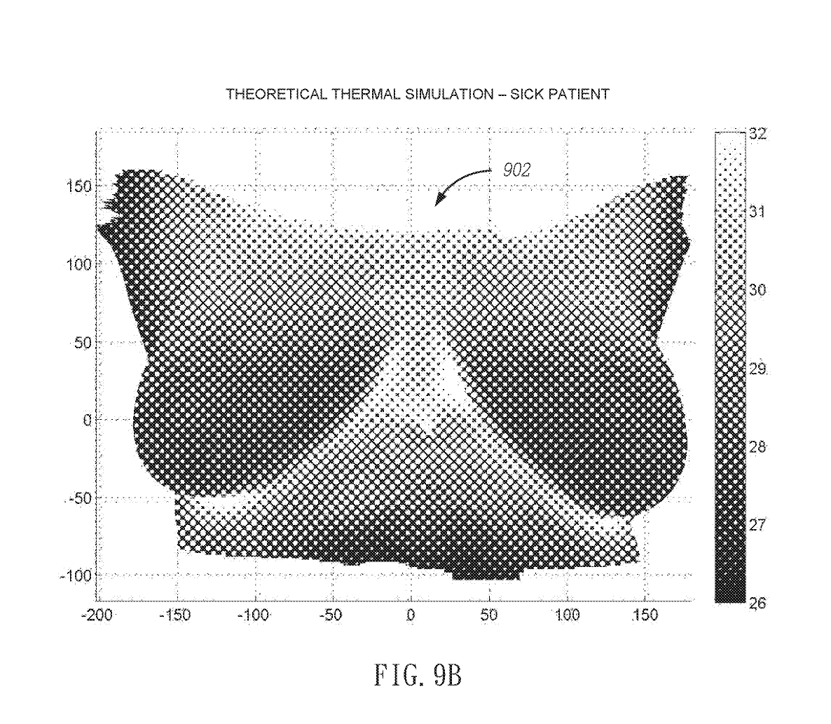

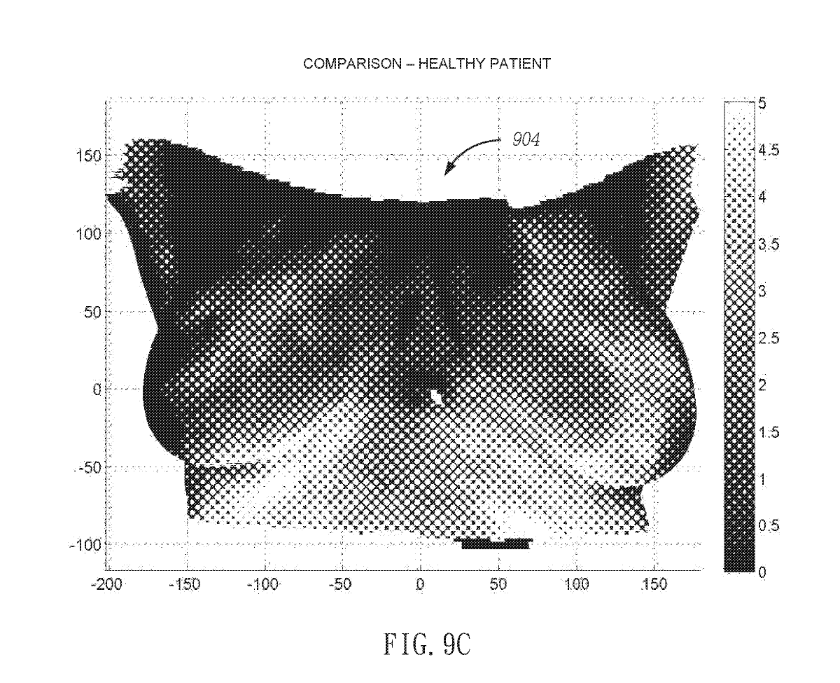

Reference is now made to FIGS. 9A, 9B and 9C, which show pictorial views of a spatial thermal representation 900, a theoretical thermal simulation 902 and a comparison 904, respectively—all of a sick subject having breast abnormalities (e.g. tumors). Representation 900 and simulation 902 are shown as a heat map, wherein darker areas mean lower temperature whereas lighter areas mean higher temperature. The heat map is displayed on a scale of 26 or 27 to 34 degrees Celsius.

As can be observed in FIG. 9A, spatial thermal representation 900 includes areas of different temperature which are randomly located, sized and shaped—as acquired in reality by the present imaging device. In contrast, theoretical thermal simulation 902 of FIG. 9B is shown with smoother and far more arranged temperature gradients. That is, theoretical thermal simulation 902 represents a mathematical model of temperature gradients of a 3D reconstruction of that patient's breasts.

Comparison 904 of FIG. 9C shows temperature differences between spatial thermal representation 900 and theoretical thermal simulation 902. As can be observed, the majority of the area of comparison 904 is indicative of a temperature difference of approximately 2.5 to 5 degrees Celsius, whereas the remaining area indicates a temperature difference of approximately 0 to 1.5 degrees Celsius. Namely, comparison 804 indicates that the temperature differences are significant.

In sum, significant temperature differences between a spatial thermal representation and a theoretical thermal simulation, both in 3D, may be indicative, where they appear, of an abnormality such as one or more tumors. In some embodiments, a user may set a temperature difference threshold, above which the method alerts of the possible existence of an abnormality. The threshold may optionally pertain also to a size of an area of that temperature difference, to filter out areas which are either too small or too large to represent a real abnormality.

It should be understood that the above mentioned embodiments may be applied for determining the likelihood of the presence of a thermally distinguishable object in any object, based on said comparisons.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An imaging method comprising:
receiving a spatial thermal representation of a curved body section, wherein the spatial thermal representation comprises thermal data associated with spatial data; and
generating a theoretical thermal simulation of the curved body section,
wherein said generating of the theoretical thermal simulation comprises:
based on the spatial data of the representation, defining a reference point or isothermal surface in the body section, and determining distances of points on the body's surface to said reference point or isothermal surface; and
calculating a thermal map based on said distances and on predetermined thermodynamic logic of the curved body section or a type thereof.

2. The method according to claim 1, further comprising comparing the spatial thermal representation and the thermodynamic logic.

3. The method according to claim 2, further comprising detecting an abnormality in the curved body section, wherein said detecting is based on said comparing of the spatial thermal representation and the thermodynamic logic.

4. The method according to claim 3, further comprising back-solving a parameter of the abnormality inside the curved body section.

5. The method according to claim 4, wherein said back-solving comprises:
generating a plurality of additional theoretical thermal simulations of a theoretical tumor inside the curved body section, wherein, in each simulation of the plurality of additional theoretical thermal simulations, a parameter of the theoretical tumor is adjusted; and
comparing the spatial thermal representation and the results of the plurality of additional theoretical thermal simulations, to determine which simulation of the plurality of additional theoretical thermal simulations is closest to the representation.

6. The method according to claim 4, wherein the parameter of the abnormality is selected from the group consisting of: a location of the abnormality inside the curved body section, a size of the abnormality, a shape of the abnormality, and a type of the abnormality.

7. The method according to claim 3, wherein said spatial thermal representation is based, at least in part, on a cold stress test.

8. The method according to claim 7, wherein the predetermined thermodynamic logic is based, at least in part, on a theoretical cold stress test.

9. The method according to claim 1, wherein the predetermined thermodynamic logic of the type of the curved body section is computed based on a healthy subject.

10. The method according to claim 1, wherein the curved body section comprises one or more breasts.

11. An imaging system comprising:
an imaging device; and
a hardware data processor configured to:
(a) generate a spatial thermal representation of a curved body section, wherein
the spatial thermal representation comprises thermal data associated with spatial data,
(b) define a reference point or isothermal surface in the body section, and determine distances of points on the body's surface to said reference point or isothermal surface
based on the spatial data of the representation; and
(c) calculate a thermal map based on said distances and on predetermined thermodynamic logic of the curved body section or a type thereof.

12. The imaging system according to claim 11, wherein said hardware data processor is further configured to compare the spatial thermal representation and the theoretical thermal simulation.

13. The imaging system according to claim 12, wherein said hardware data processor is further configured to detect an abnormality in the curved body section, wherein said detect is based on said comparing of the spatial thermal representation and the thermodynamic logic.

14. The imaging system according to claim 13, wherein said hardware data processor is further configured to back-solve a parameter of the abnormality inside the curved body section.

15. The imaging system according to claim 14, wherein said back-solve comprises:
generating a plurality of additional theoretical thermal simulations of a theoretical tumor inside the curved body section, wherein, in each simulation of the plurality of additional theoretical thermal simulations, a parameter of the theoretical tumor is adjusted; and
comparing the spatial thermal representation and the results of the plurality of additional theoretical thermal simulations, to determine which simulation of the plurality of additional theoretical thermal simulations is closest to the representation.

16. The imaging system according to claim 14, wherein the parameter of the abnormality is selected from the group consisting of: a location of the abnormality inside the curved body section, a size of the abnormality, a shape of the abnormality and a type of the abnormality.

17. The imaging system according to claim 13, wherein said spatial thermal representation is responsive to a cold stress test, thereby enhancing a contrast between the abnormality and a normal tissue adjacent to the abnormality.

18. The imaging system according to claim 17, wherein the predetermined thermodynamic logic is under an influence of a theoretical cold stress test.

19. The imaging system according to claim 11, wherein the predetermined thermodynamic logic of the type of the curved body section is computed based on a healthy subject.

20. The imaging system according to claim 11, wherein the curved body section comprises one or more breasts.

21. The imaging system according to claim 11, wherein said imaging device comprises a thermal imaging device and a visible light imaging device.

22. An imaging method comprising:
receiving spatial data of a curved body section;
defining a reference point in the body section;
determining distances of points on the body's surface to said reference point;
calculating a thermal map based on said distances, and
on predetermined thermodynamic logic of the curved body section or a type thereof.

23-30. (canceled)

31. The method according to claim 22, wherein the predetermined thermodynamic logic of the type of the curved body section is computed based on a healthy subject.

32. The method according to claim 22, wherein the curved body section comprises one or more breasts.

33. An imaging system comprising:
an imaging device; and
a hardware data processor configured to generate spatial data of a curved body section, to define a reference point in the body section, to determine distances of points on the body's surface to said reference point, and to calculate a thermal map based on said distances and on predetermined thermodynamic logic of the curved body section.
